SEQUENCE LISTING (3 sequences)

SEQ ID NO: 1 - protein, 32 aa, Homo sapiens
PEAEDLQVGQVELGGGPGAGSLQPLALEGSLQ

SEQ ID NO: 2 - protein, 32 aa, Monkey
PEAEDPQVGQVELGGGPGAGSLQPLALEGSLQ

SEQ ID NO: 3 - protein, 30 aa, Pig
PEAENPQAGAVELGGGLGGLQALALEGPPQ

(19) United States
(12) Patent Application Publication
(10) Pub. No.: US 2009/0214482 A1
(43) Pub. Date: Aug. 27, 2009

(54) Transgenic Mammals Expressing Human Preproinsulin

(75) Inventors: William Beschorner, Omaha, NE (US); Patrick E. Kudlacek, Omaha, NE (US)

Correspondence Address: BANNER & WITCOFF, LTD., 1100 13th STREET, N.W., SUITE 1200, WASHINGTON, DC, 20005-4051, US

(73) Assignee: XIMEREX, inc., Blair, NE (US)

(21) Appl. No.: 11/918,281

(22) PCT Filed: Apr. 11, 2006
(86) PCT No.: PCT/US06/13428

§ 371 Date: Jan. 28, 2009

Related U.S. Application Data
(60) Provisional application No. 60/670,653, filed on Apr. 13, 2005.

(51) Int. Cl. A61K 35/12; A01K 67/027; C07H 21/04; C12N 5/10

(52) U.S. Cl. 424/93.7; 800/14; 800/17; 536/23.5; 536/23.2; 435/366

(57) ABSTRACT

Transgenic mammals which express human preproinsulin, methods and reagents for producing the transgenic mammals, and therapeutic methods of providing patients with insulin and C-peptide using tissues and cells from the transgenic mammals.

This application claims the benefit of and incorporates by reference co-pending provisional application Ser. No. 60/670,653 filed Apr. 13, 2005.

### FIELD OF THE INVENTION

The invention relates to transgenic mammals which express human preproinsulin, methods and reagents for producing the transgenic mammals, and therapeutic methods of providing patients with insulin using tissues and cells from the transgenic mammals.

### BACKGROUND OF THE INVENTION

As many as 1.2 million Americans have type I diabetes mellitus. World-wide, as many as 10 million persons may be so affected. Type I diabetes is caused by the autoimmune destruction of insulin-producing beta cells. There are two major consequences: loss of control of glucose metabolism and chronic complications, including vascular disease. Careful monitoring of blood glucose and administration of exogenous insulin prevents the major complications of hyperglycemia, such as ketoacidosis. Nonetheless, most diabetics eventually develop chronic complications, especially vascular disease, leading to renal failure, impairment of circulation, blindness, and neurological disease.

Five years ago, the potential to cure type I diabetes came much closer to reality. Using adequate numbers of human cadaveric islets and modest immune suppression with agents that were not toxic to the islets, Shapiro et al. achieved prolonged engraftment in diabetic patients with prolonged insulin free follow-up (Shapiro et al., Lancet 362, 1242, 2003). Some of the original recipients still do not require exogenous insulin. Moreover, there is evidence that the allografts provide protection against the chronic complications of diabetes (Fiorina et al., Transplantation 75, 1296-301, 2003).

The success of this inspiring accomplishment is diminished by two major problems. First, the number of human donors available can provide grafts for only a small portion of those in need (less than 1%). Second, the need for chronic immune suppression makes this technology unfeasible for most children, for whom the benefit of the islet transplant must be weighed against chronic complications of the chemotherapy.

Islets, islet cell clusters, or beta cells from mammalian donors such as pigs could resolve the severe shortage of human donors. Pig insulin differs from human insulin by only one amino acid. However, the C-peptide, which connects the A and B chains of insulin differs greatly between species.

C-peptide was initially believed to be an inactive peptide. Evidence is now accumulating that C-peptide is not inactive but rather is integral in protecting the host from vascular complications of diabetes. C-peptide binds to cell surfaces through G-protein coupled receptors. When administered to diabetic animals, it improves glomerular filtration, reducing excretion of albumin (Wahren et al., Curr. Diab. Rep. 1, 261-66, 2001, Wahren, Clin. Physiol Funct. Imaging 24, 180-89, 2004). C-peptide enhances blood flow in vessels by stimulating nitrous oxide release by endothelial cells. C-peptide also has non-vascular effects, such as with renal tubules and motor neurons. C-peptide administered to diabetic rats prevented the chronic complications of diabetes, including glomerular basement membrane thickening and diabetic glomerulosclerosis (Samnegard et al., Nephrol Dial. Transplant. 20, 532-38, 2005).

The amino acid sequence for human and monkey C-peptides are nearly identical with only one amino acid substitution. The porcine C-peptide, though, has 11 amino acid differences, including a 2 amino acid gap in the pig (Ido et al., Science 277, 563-566, 1997).

(SEQ ID NO: 1)

Human (31) EAEDLQVGQVELGGGPGAGSLQPLALEGSLQ

(SEQ ID NO: 2)

Monkey (31) EAEDPQVGQVELGGGPGAGSLQPLALEGSLQ

(SEQ ID NO: 3)

Pig (29) EAENPQAGAVELGGGLGG_LQALALEGPPQ

The homology differences could have several adverse effects, including differences in clearance from the blood (Wennberg et al., Cell Transplant. 10, 165-73, 2001), binding to the endothelial G-protein coupled receptors, and exacerbation of chronic vascular complications. The disparate form of porcine C-peptide could also lead to anti-C-peptide antibodies in the human recipient.

In a model demonstrating the contribution of C-peptide to glucose induced blood flow through skin capillaries, porcine C-peptide enhanced blood flow only 20% as much as human C-peptide.

There is a need in the art for alternate sources of pancreatic islets.

### BRIEF DESCRIPTION OF THE DRAWINGS

FIG. 1. Drawings of genetic construct encoding human preproinsulin, the enzyme neomycin phosphotransferase (which confers neomycin resistance), and chloramphenicol acetyltransferase (CAT) for homologous recombination into the porcine genome. FIG. 1A, construct with two promoters. FIG. 1B, construct with one promoter.

FIG. 2. Graph showing intravenous glucose tolerance tests for a diabetic macaque, a normal macaque, and two porcine islet cell cluster transplant recipients.

FIG. 3. Photomicrograph showing Immunohistochemistry of liver demonstrating insulin secreting cells.

FIG. 4. Graph showing results of quantitative RT-PCR to identify porcine endogenous retrovirus (PERV), demonstrating absence of infection in the chimeric recipients.

### DETAILED DESCRIPTION OF THE INVENTION

The invention provides genetic constructs which can be used to create transgenic mammals which produce human preproinsulin and can therefore provide tissues and cells which can be transplanted into human patients to treat diabetes, pancreatic cancer, or chronic pancreatitis.

Methods are available which can tolerize prospective transplant recipients to donor tissues. See, e.g., U.S. Pat. No. 6,923,959 and U.S. Pat. No. 6,060,049. In a preferred method, lymphocytes from the recipient are grown within fetal mammals destined to become xenograft donors. The lymphocytes become tolerant to the mammalian tissues. This tolerance is later transferred back to the recipient by infusing regulatory lymphocytes from the chimeric donor mammal back to the recipient (Beschorner et al., Transplant. Proc. 28, 648-49; 1996, Beschorner et al., Annals of Surgery 237, 265-72, 2003). Tissue accommodation is also achieved within the donor mammal before transplantation. Using this technique, two pig islet xenografts in rhesus macaques survived 98 and 222 days without post-transplant immune suppression. In both animals, the cause of death was unrelated to the transplant. Both macaques had functional islet xenografts when they died.

### Genetic Constructs

Genetic constructs of the invention comprise a coding sequence for human preproinsulin under the control of a non-human mammalian preproinsulin promoter (e.g., pig, rat, cow) and can comprise one or more coding sequences for a selectable marker and/or an inert marker protein. Expression of the inert marker protein preferably is under control of the same mammalian preproinsulin promoter (i.e., the coding sequences are in-frame).

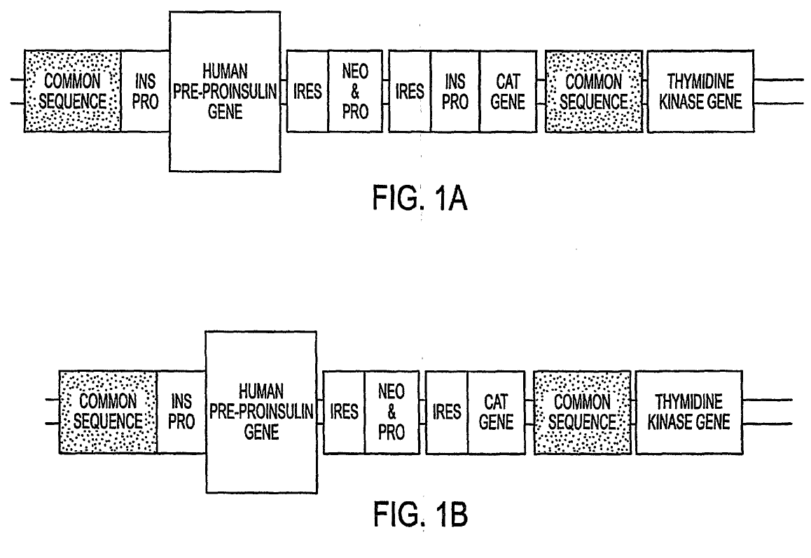

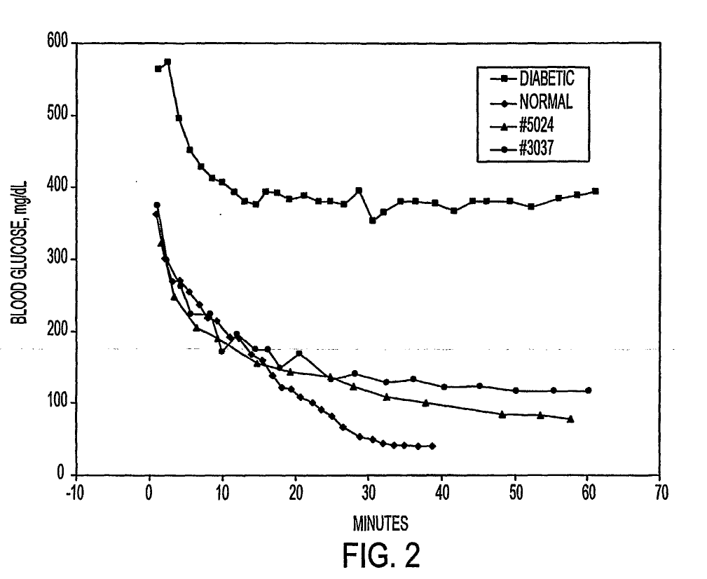

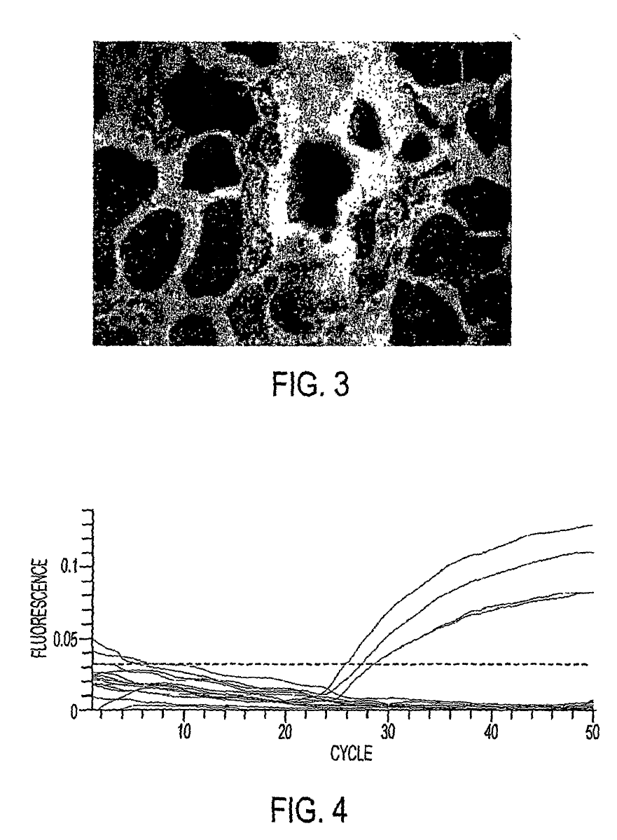

Genetic constructs of the invention can be made using standard recombinant DNA techniques well known in the art. See also Example 1. Two embodiments are shown in FIG. 1. Other options for such constructs are described below. For example, the neomycin resistance gene can be replaced with any positive selection gene which permits selection of cells containing the construct. The thymidine kinase gene can be replaced with any suicide gene which permits elimination of cells which have not undergone homologous recombination. One or more preproinsulin promoters can be used in a single construct. If desired, IRES genes can be included as is known in the art. The human preproinsulin gene could be replaced with a gene having less than complete amino acid homology with human preproinsulin but greater homology than the native mammalian preproinsulin gene.

Human Preproinsulin

The amino acid sequence of human preproinsulin is known in the art. See, e.g., Ulrich et al., Science. 1980 Aug. 1; 209(4456):612-5; Sures et al., Science. 1980 Apr. 4; 208(4439):57-9; Bell et al., Nature. 1979 Nov. 29; 282(5738):525-7; Georges et al., Gene. 1984 February; 27(2):201-11; Narang et al., Can J Biochem Cell Biol. 1984 April; 62(4):209-16; Wang et al., Sheng Li Xue Bao. 2003 Dec. 25; 55(6):641-7; O'Driscoll et al., In Vitro Cell Dev Biol Anim. 2002 March; 38(3):146-53; and Shaw et al., Endocrinol. 2002 March; 172(3):653-72. Any nucleotide sequence encoding human preproinsulin, including naturally occurring forms, can be used, as long as the naturally occurring forms of human preproinsulin do not have the same sequence as the transgenic mammal (e.g., pig) preproinsulin. In some embodiments, nucleotide sequences are used which encode preproinsulin which is partially homologous with the human protein (e.g., which has an amino acid sequences which is closer to the human peptide than to the native donor mammal protein).

Preproinsulin Promoter

The nucleotide sequence of the pig preproinsulin promoter is known in the art (Han & Tuchm, Comp Biochem Physiol B Biochem Mol. Biol. 2001 May; 129(1):87-95). The sequences of the mouse and rat promoters also are known (Welsh et al., Mol. Med. 5, 169-80, 1999). Promoters from one mammalian species may be used in a transgenic mammal of another species. For example, transgenic mice produce physiologic amounts of beef insulin under control of the human promoter (Poplonski et al., Eur. J. Immunol. 26, 601-09, 1996).

Inert Marker Proteins

Transplants of cells and cell clusters are challenging to monitor for rejection. For example, islets and islet cell clusters injected into the portal vein become widely dispersed throughout the liver. Thus, a liver biopsy would most likely miss the transplanted cells. A decrease in mammalian C-peptide levels or insulin can be helpful in detecting rejection, but these levels could also reflect the patient's metabolism of these proteins. Because antibodies are cross reactive between human and other mammalian forms of insulin and C-peptide, tests are often unreliable in quantifying the insulin and C-peptide produced by the xenograft. Furthermore, because the invention can produce insulin and C-peptide with identical or close homology with human isoforms, it would be impossible or more difficult to quantify the insulin or C-peptide produced by the graft. Islet mass is currently estimated with the arginine stimulation test (Larsson & Ahren, Diabetologia 41, 772-77, 1998). This test is stressful to the patient. Moreover, the outcome of the test reflects islet function as well as mass, so the results do not distinguish between the mass of residual islets in the patients and transplanted islets.

To solve these problems, according to the invention a gene for an inert marker protein can be inserted into the transgenic mammal under the control of the same preproinsulin promoter. The inert marker protein can be any soluble, inert protein not present in the recipient. Suitable inert marker proteins include, but are not limited to, chloramphenicol acetyltransferase (CAT), human chorionic gonadotrophin (hCG), soluble tumor necrosis factor receptor, β-galactosidase, and the like. CAT is preferred. It is secreted by the beta cells in a consistent manner, can be readily quantified in the serum and urine, and has proved useful for measuring cell mass and monitoring gene therapy. See Dass & Burton, Biother. Radiopharm 17, 501-05, 2002.

Selectable Markers

A wide variety of gene products can be used for selective identification, isolation, or propagation of transfected cells. A selectable marker can confer resistance to an antibiotic or drug. Genes which confer resistance to particular antibiotics, such as kanamycin, hygromycin, neomycin, gentamicin A, and gentamicin B, are useful as selectable markers. Alternatively, the selectable marker can be a conditionally toxic gene, for example Herpes simplex virus thymidine kinase (HSV-TK), which is used in conjunction with tk cell lines; the CAD gene, which is used in conjunction with CAD-deficient cells; and the mammalian hypoxanthine-guanine phosphoribosyl transferase (hprt) gene, which is used in conjunction with hprt⁻ cell lines. In some embodiments, more than one selectable marker is used (e.g., neomycin resistance and TK). A review of the use of selectable markers in mammalian cell lines is provided in Sambrook et al., Molecular Cloning: A Laboratory Manual, 2nd ed., Cold Spring Harbor Laboratory Press, New York (1989) pp. 16.9-16.15.

### Transgenic Mammals

The genetic constructs described above can be used to produce transgenic mammals by any method known in the art. These methods include, but are not limited to, microinjection, embryonic stem (ES) cell manipulation, electroporation, cell gun, transfection, transduction, retroviral infection, etc. Transfected fetal mammalian fibroblasts can be transferred into enucleated mammalian oocytes by nuclear transfer methods, activated and implanted into the oviducts of surrogate mammals. See Examples 2 and 3, below. See also U.S. Pat. No. 5,922,854 and US 2005/0260746. Species of genetic constructs may be introduced individually or in groups of two or more types of construct.

Of the 39 major orders of the class Mammalia, five orders are particularly suitable for use as transgenic mammals: primates (e.g., chimpanzees, gorillas, lemurs), artiodactyls (e.g., pigs, sheep, goats, and cows), carnivores (e.g., dogs, cats), rodents (e.g. rats, mice), and lagamorphs (e.g., rabbits and hares).

In some embodiments the transgenic mammals are hemizygous, with one chromosome expressing the human preproinsulin gene and CAT and the other chromosome expressing the native mammalian preproinsulin. These transgenic mammals provide native insulin and C-peptide for themselves and human insulin and C-peptide in a human recipient. Within the transgenic mammal or the human, the irrelevant C-peptide would be less effective in binding to receptors and would be cleared more rapidly than the relevant C-peptide.

Another embodiment involves homozygous transgenic mammals expressing the human preproinsulin on both chromosomes and the CAT gene on one or both chromosomes. The human C-peptide provides protection for such mammals. There is a major advantage of using islets from homozygous mammals, because the transplanted islets would not produce any donor insulin or C-peptide. This is particularly important when the transgenic mammal is a pig because some diabetics treated with porcine insulin have developed anti-insulin antibodies. Because of the considerable amino acid disparity between human and porcine C-peptide, antibodies would likely develop to the C-peptide as well.

Homozygous transgenic mammals can be produced from the hemizygous mammals by either breeding male and female hemizygous mammals or by transfecting fibroblasts from a hemizygous mammal and selecting the transfected cells that have lost the mammalian preproinsulin. The viability of pig islets and rejection of pig islets, for example, have been monitored in the past by the presence of porcine C-peptide. Because mammalian C-peptide would no longer be produced by islets from the homozygous mammals, an alternate marker of the islets, such as CAT, is a preferred embodiment for monitoring engraftment and rejection.

Transgenic Pigs

Transgenic pigs are a particularly useful embodiment of the invention. The “pre” peptide is strongly conserved between pigs and humans, and the human “pre” signal functions appropriately within the pig, activating the human proinsulin. Using pigs as islet donors is an attractive near term solution to the donor shortage. The physiology of most pig organs is similar to that of humans. Indeed, porcine insulin was the standard insulin before recombinant human insulin became available. Pig and human insulin differ by only one amino acid and have very similar pharmacokinetics. Thorsteinsson et al., Eur. J. Clin. Pharmacol. 33, 173-78, 1987. In pigs, the islets maintain the same level of glucose as humans (100 mg %). In contrast, non-human primates maintain a lower glucose level.

The glutamic acid decarboxylase expressed on porcine islets is not affected by antibodies from subjects with autoimmune diabetes. Koulmanda et al., Xenotranpl. 10, 178-84, 2003; Rowley et al., Clin. Exp. Immunol. 106, 323-28, 1996. Thus, pig islets may also be more resistant to the autoimmune reactions of type I diabetes than human islets.

Moreover, the ability to induce immune tolerance to pig xenografts without a period of immune deficiency makes porcine xenografts highly competitive with both allogeneic tissue transplants and islets derived from ESC.

### Transgenic Mammalian Tissues and Cells for Transplantation

Transgenic mammals of the invention can be used to provide tissues and cells for therapeutic treatment of human diabetes. Methods of obtaining pancreatic tissue, including islets, and making preparations of islet cells and beta cells are well known in the art. See Examples 4 and 5. See also U.S. Pat. No. 6,946,293, U.S. Pat. No. 6,815,203, U.S. Pat. No. 6,815,203, U.S. Pat. No. 6,783,964, U.S. Pat. No. 6,303,355, U.S. Pat. No. 5,773,255, U.S. Pat. No. 4,935,000, US 2004/0033216, US 2003/0129173, and US 2003/0082155.

Methods of obtaining adult and embryonic stem cells and differentiating them into functional beta cells also are well known. See Example 6. See also U.S. Pat. No. 6,326,201, US 2006/0040387, and US 2005/0054102.

Tissues and cells obtained from transgenic mammals of the invention can be placed in transport media appropriate for transplantation into patients. The compositions of such media are well known to those familiar with the field.

### Therapeutic Methods

Methods of transplanting islet preparations and beta cells for treating diabetes are known in the art. See, e.g., U.S. Pat. No. 6,703,017. Any of these methods can be used to transplant islet preparations and beta cells from transgenic mammals of the invention. See also Examples 4, 5, and 6.

Transplanted patients can be monitored for glucose, insulin, and C-peptide levels, and, optionally, the expression of the inert marker protein. Glucose tolerance tests can be performed periodically. The recipient can be evaluated clinically and histologically for evidence of chronic complications of diabetes. Using methods of the invention, a patient's dependence on an exogenous source of insulin is decreased.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Gene Construct Containing Human Preproinsulin and Optionally Chloramphenicol Acetytransferase

The genetic construct illustrated in FIG. 1 contains, between homologous porcine/human genomic sequences, a coding sequence for human preproinsulin in frame with a coding sequence of a neomycin resistance gene and a coding sequence for chloramphenicol acetyltransferase and, down-stream of a porcine homologous region, a sequence for thymidine kinase. DNA sequences for use in making the genetic constructs can be isolated and ligated through the use of standard recombinant DNA techniques, such as PCR and the use of restriction and ligase enzymes. A circular vector containing the DNA sequences is transfected into E. coli for proliferation and sequencing. Upon completion of the genetic construct and confirmation of the sequence, the genetic construct is purified and prepared for transfection into fetal pig fibroblasts.

Fetal pigs (35 days gestation) are obtained by Caesarian section from pregnant sows. The three centimeters long fetuses are minced into 1-mm³ pieces and 0.5% trypsin is added. After incubation for half an hour, the isolated cells are filtered. After two washes in PBS the cells are seeded in 170 cm² flasks with DMEM plus 10% fetal calf serum. After one week, when the cultures are confluent, cultures are split into 3 flasks. This is counted as a passage 1. This sub-culture procedure is repeated twice. The gender of the fetal pig fibroblasts (FPF) of passage 3 is determined by cytogenetics analysis. The cells are frozen at −150° C.

Between 8 to 10 million FPF cells are transfected by electroporation using the following method. FPF are diluted in 0.4 ml of PBS, poured into a 0.4 cm electroporation cuvette, and placed on ice for 10 minutes. Linearized plasmid (10-20 μg) is added to the cell mixture. The electroporator is set at 0.300 mV and 0.950 pFahr, and the cells are shocked for 31 μsec. Following electroporation, cells are seeded immediately onto a 10 mm plastic dish containing DMEM with 10% FCS. After 48 hours of incubation the medium is changed to a DMEM containing neomycin. After three weeks in culture, colonies are scored for transfection with PCR. Colonies isolated this way are transferred to a 24-well-plate. Homologous recombination is established by incubating the selected cells for 5 days in gancyclovir, which kills the cells which include the TK gene. The surviving cells are trypsinized and either frozen in DMSO or further amplified.

### Example 2

### Production of Transgenic Hemizygous Pigs which Express Human Preproinsulin

Preparation of Recipient Oocytes

Oocytes from sow ovaries are purchased from BoMed, Inc. (Madison, Wis.). The oocytes are matured in TCM 199-Hepes, supplemented with 5 μg/ml insulin, 10 ng/ml EGF, 0.6 mM cysteine, 0.2 mM Na-pyruvate, 3 μg/ml FSH 25 ng/ml gentamicin and 10% porcine follicular fluid. They are shipped in maturation medium over night. Oocytes derived from gilts are matured in defined protein medium (TCM 199 supplemented with 0.1% PVA 0.1 mg/ml cysteine, 10 ng/ml EGF, 0.91 mM Na-pyruvate, 3.05 mM D-glucose, 0.5 μg/ml FSH, 0.5 μg/ml LH, 75 μg/ml penicillin, and 50 μg/ml streptomycin).

Nuclear Transfer

After 44 h of oocyte maturation, oocytes are freed from cumulus cells by vigorous vortexing for 4 min in TL-Hepes supplemented with 0.1% PVA and 0.1% hyaluronidase. Cumulus-free (denuded) oocytes are enucleated by aspirating the first polar body and adjacent cytoplasm in enucleation medium with a glass pipette 30 μm in diameter (Lai & Prather, Reprod. Biol. Endocrinol. 1, 82, 2003). The donor cells are injected directly into the perivitelline space of the oocyte. Injected oocytes are placed between 0.2 mm diameter platinum electrodes 1 mm apart in fusion/activation medium. Fusion/activation is induced with 2 DC pulses (1 sec interval) of 1.2 kV/cm for 30 μsec on a BTX ECM 830 (BTX, San Diego, Calif.). The medium used for enucleation is tissue culture medium (TCM) 199 supplemented with HEPES, 0.3% BSA, and 7.5 μg/mL cytochalasin B (CB), and the medium for injection is the same medium without CB. The medium used for fusion and activation consists of 0.3 M mannitol, 1.0 mM CaCl₂, 0.1 mM MgCl₂ and 0.5 mM HEPES.

Embryo Transfer

The surviving embryos (intact plasma membrane) are selected for transfer into an oviduct of the surrogate sows after culture for 18-22 h. Potential domestic surrogate sows are hormonally cycled to be in estrus and are heat checked. Between 50-180 nuclear transfer-derived embryos are transferred into recipient oviducts 5-18 hours following the onset of estrus. Anesthesia is initially induced with ketamine and maintained with isoflurane. While in a dorsal recumbent position the surrogates are aseptically prepared for surgery, and a 10 cm incision is made into the abdominal midline to expose the oviduct. Embryos are transferred from culture medium into the manipulation medium and loaded into a Tomcat catheter (3½ Fr.: Sherwood medical, St. Louis, Mo., USA) attached to a 1 ml syringe. Embryos are placed in the ampullar region of oviduct by inserting 5 cm of the catheter through the ovarian fimbria and into the ampulla. The surrogate sow receives Buprenorphine (0.1 mg/kg i.m.) for analgesia.

Screening of Offspring

Oligonucleotide primers specific for each construct are commercially synthesized. Ear biopsies and blood are digested, and DNA is extracted. DNA samples from each animal are subjected to PCR. Briefly, PCR is performed in 50 μl reactions and 30 cycles of 94° C. for 2 minutes, 50° C. for 2 minutes, and 72° C. for 2 minutes. Water is used as a positive control, and porcine genomic DNA is used as a negative control. Reactions are run out on an agarose gel to determine if amplicons are the appropriate size.

The transgenic pigs are challenged with a glucose tolerance test. Serum and urine are screened for human insulin and C-peptide. Islets in cell culture are challenged and the production of human insulin and C-peptide assessed.

### Example 3

### Production of Transgenic Homozygous Transgenic Pigs with the Human Preproinsulin and CAT Marker

Homozygous transgenic pigs expressing only the human preproinsulin are produced either by breeding male and female transgenic pigs produced by methods in Example 2 or by transfecting and cloning cells, such as fetal pig fibroblasts, from hemizygous pigs produced in Example 2. The resulting offspring or clones are screened by PCR for the presence of the new gene construct (including the CAT gene) and the absence of the smaller native gene (without the CAT gene). The homozygous cells are then transferred into enucleated porcine oocytes, activated, and grown into transgenic offspring in surrogate sows.

The transgenic pigs are challenged with a glucose tolerance test. Serum and urine are screened for human insulin and C-peptide. Islets in cell culture are challenged, and the production of human insulin and C-peptide assessed.

### Example 4

### Use of Pancreatic Tissue from Transgenic Pigs Expressing Human Preproinsulin for Transplantation

Pancreas transplants are carried out for patients with diabetes and for patients in which their native pancreas is no longer functional or has been removed, such as with chronic pancreatitis, or pancreatic carcinoma. The donor pig is a transgenic pig expressing human preproinsulin. Preferably the donor pig is homozygous and expresses the CAT gene as well. The donor pig is anesthetized and maintained on a gas anesthesia machine. The abdomen is thoroughly cleaned and prepared with Betadine. A midline incision is made on the abdomen. Pancreatic tissue is identified and removed for preparation for transplantation. It is perfused with transport media such as University of Wisconsin solution or Eurocollins solution.

Procedures known to those skilled in the art are undertaken to prevent rejection of the xenograft. In some embodiments, chimeric pig donors which contain lymphocytes from the patient are used. The lymphocytes are tolerant to the pig tissue and are transferred back to the recipient (e.g. from the chimeric pig spleen). Alternately, the patient is treated with chemotherapy and other measures before and after transplantation to prevent rejection.

The patient may also receive another organ transplant at the same time. For example, patients with diabetes and diabetic nephropathy may receive a kidney from the same donor pig.

Rejection is monitored by standard methods known to those familiar with the field. These include biopsies of the pancreas and other transplant tissues such as the kidney. Alternately, the patient can be followed for levels of CAT in the serum and in the urine. The levels define the function of the graft. When they fall, that can indicate rejection of the pancreatic islets.

The patient is monitored for glucose, insulin, C-peptide, and CAT levels. Glucose tolerance tests are performed periodically. The recipient is evaluated clinically and histologically for evidence of chronic complications of diabetes.

### Example 5

### Use of Islets or Beta Cells from Transgenic Pigs Expressing Human Preproinsulin for Transplantation

As in Example 4, transgenic pigs that express human preproinsulin are used as islet donors, preferably homozygous transgenic pigs. Known methods are used to isolate islet cell clusters from fetal or neonatal pigs or islets from mature pigs. For example, porcine islet cell clusters from young pigs are isolated as follows. Pancreatic tissue, preferably an intact pancreas or a portion of a pancreas containing the pancreatic duct, is obtained from the donor pig as described above. The tissue is chilled and placed in transport medium. Within a laminar-flow hood, the tissue is transferred into a shallow chilled dissecting pan. The pancreatic duct is cannulated with a blunt needle and infused with chilled collagenase solution until the pancreas is well distended. The distended pancreas and collagenase are transferred into a sieve basket (450μ mesh) within a heated digestion chamber (37° C.) designed to allow continuous removal of digested cells and cell clusters. The pancreas is monitored for tissue dissociation. When tissue particles become evident, digesting tissue is stirred to release cell clusters. The stirring is halted intermittently to allow cell clusters to sediment through the sieve for collection. The cell clusters are collected and washed until the entire pancreas has been digested. The washed cell clusters are transferred into long-term tissue culture vessels with appropriate growth-promoting medium.

During culture, the number of cells that stain red with dithizone (indicating the zinc granules that correspond to β-cells) will steadily increase, from a few percent to more than 30 percent. During culture there is also a profound reduction in the proportion and total amount of exocrine tissue present.

The resulting islet cell clusters or islets are transplanted into the patient (1,000 to 30,000 islet equivalents/kg), for example, by infusion into the portal vein of the recipient (Scharp et al., Cell Transplant. 1, 245-54, 1992).

The patient is monitored for glucose, insulin, and C-peptide levels. In addition, the serum and urine is monitored for a fall in the CAT levels, which would suggest rejection of the islets. Interventions are performed as indicated, such as increased immune suppression. The patient is evaluated periodically for clinical or histologic evidence of chronic complications of diabetes.

Measures such as those described in Example 4 are taken to prevent rejection of the islet cell clusters or islets. Alternatively, the islet cell clusters or islets can be encapsulated within devices by semipermeable membranes that are permeable to insulin, C-peptide, and glucose, but are not permeable to antibodies or white cells. Alternately, the islet recipients may be treated with immune suppressing agents to prevent acute rejection of the islets and beta cells.

### Example 6

### Generation of Beta Cells from Progenitor Cells or Embryonic Stem Cells of Transgenic Pigs Expressing Human Preproinsulin

Instead of digesting the pancreas from the donor pig, alternately islet progenitor cells, adult stem cells, or embryonic stem cells are cultured under the appropriate growth factors for differentiation into beta cells or islets. The methods are known to those familiar with the field. The cells are obtained from embryos, fetal or neonatal pigs, or from mature pigs derived from the transgenic pigs that express the human preproinsulin.

For example, porcine embryonic stem cells can be isolated from blastocysts following in vitro fertilization. The stem cells are then cultured under conditions that produce functional beta cells. The beta cells are transplanted into the diabetic recipient. Measures are taken to prevent rejection.

The patient is monitored for glucose, insulin, C-peptide, and CAT levels. Glucose tolerance tests are performed periodically. The recipient is evaluated clinically and histologically for evidence of chronic complications of diabetes.

### Example 7

### Surrogate Tolerogenesis for Porcine Islet Cell Cluster Xenotransplants

Pig islets are vigorously rejected by humans and by non-human primates (Sandrin & McKenzie, Immunol. Rev. 141, 169-90, 1994; Groth et al., Transplant. Proc. 28, 538-39, 1996.). Islets produced from fetal pigs were transplanted into diabetic patients given standard immune suppression. Though transient engraftment was observed, eventually the patients rejected the islets. This example demonstrates the feasibility of chimeric donor pigs for preventing rejection of pig islets in rhesus macaques.

Methods

Bone marrow from rhesus monkeys was processed and injected into fetal pigs at 45 days gestation using ultrasound guidance. PCR assays were developed using primers designed from the human genomics library to detect human or rhesus macaque cells within pigs. These detected the alpha-1,3 galactosyltransferase pseudogene and CMP-N-acetylneuraniminic acid hydroxylase. The sensitivity was greater than 1 cell in 10,000.

After the pigs were born, they were screened for chimerism (monkey cells) using a PCR assay and flow cytometry. Accommodation was assessed by incubating the pig lymphocytes in titered amounts of fresh monkey serum or human serum. These studies were used to select the donor pig.

A single dose of xyclophosphamide (35 mg/kg) was given to each recipient primate three weeks prior to transplant. One week later, the pancreas and the spleen were removed from the donor pig. Pancreatic islet cell clusters were prepared by collagenase digestion. The chimeric porcine splenocytes were infused into the recipient monkeys (0.3-2×10⁹ lymphocytes/kg). The pancreas was removed, and the pancreatic islet cell clusters were subsequently infused into the portal vein (15,400 to 30,300 islet equivalents/kg).

Two macaques were made diabetic with high dose streptozotocin. Intravenous glucose tolerance tests (IVGTT) were performed. The blood glucose level was monitored from the central line. Human regular insulin was subsequently infused as needed to prevent hyperglycemia (>200 mg %). Blood was evaluated weekly.

Chimerism and Accommodation in Chimeric Donor Pigs

One to four pigs in each litter showed evidence of chimerism by PCR. Flow cytometry demonstrated up to 3% chimerism in the peripheral blood, mesenteric lymph nodes, spleens and thymus. Mixing studies demonstrated an efficiency of detection of 38%. The 3% chimerism observed in the spleen would correspond to 8% chimerism.

Chimeric pigs #5024 and #3037 demonstrated accommodation of the lymphocytes as compared with normal, non-chimeric pig lymphocytes exposed to fresh monkey or human serum and rabbit complement. The cytotoxicity titer with a known serum dropped 2 to 5 titers (4- to 32-fold). The lymphocytes also demonstrated overexpression of bcl2, an accommodation associated protein and an inhibitor of apoptosis.

TABLE 1
Prolonged Engraftment of Porcine Islet Cell Clusters
with Chimeric and Accommodated Donor Pigs

| | Pig | Pig Donor | Insulin- | Graft |
|---|---|---|---|---|
| Macaque | Donor | Accommo- | Free | Survival |
| # | Chimerism | dation | Days | (Days) |
| 5024 | Yes | Yes | 122 | 222 |
| 4027 | Yes | No | 5 | 98 |
| 3037 | Yes | Yes | 4 | 42 |
| 9273 | No | No | 0 | — |
| 6127 | No | No | 0 | — |

Sera from four control pigs showed detectable signal at 25 to 28 cycles. Serum from macaques showed no signal at 50 cycles. PERV is less than 1 in 10⁷ compared with pigs. PCR for PERV DNA in lymphocytes demonstrated signal proportional to chimeric pig cells (2%).

The macaque recipients of chimeric splenocytes and PICCs demonstrated prolonged chimerism with pig lymphocytes. The chimerism was lost in control recipients, where the donors were not chimeric or accommodated. They never demonstrated graft function. The WBC and lymphocyte counts were normal throughout the clinical course. The PICCs matured at 35 to 100 days post transplant in the experimental recipients, allowing them to be removed from exogenous insulin. Porcine insulin and C peptide was demonstrated. The graft in #5024 continued to improve with time. Glucose tolerance tests with the standard meal (4.0 gm glucose/kg) improved with time. His hemoglobin A1C dropped from 6.5 to 4.5.

The recipients were euthanized due to technical complications unrelated to the graft or immune competence. For example, #5024 developed a bile duct atresia secondary to the pancreatectomy performed for diabetes.

The pig islet xenografts initially provided partial regulation of the blood glucose levels. Later, the islets appeared to mature, providing the recipient with an insulin free regulation of glucose. The prolonged acceptance was achieved with minimal pretransplant immune suppression and no post-transplant immune suppression. The chimeric recipient macaques did not demonstrate infection with PERV.

1. A genetic construct comprising a coding sequence for human preproinsulin under control of a pig preproinsulin promoter.

2. (canceled)

3. The genetic construct of claim 1 further comprising a coding sequence for an inert marker protein under the control of the preproinsulin promoter.

4. The genetic construct of claim 1 wherein the inert marker protein is chloramphenicol acetyltransferase.

5. The genetic construct of claim 1 further comprising a selection marker.

6. The genetic construct of claim 5 wherein the selection marker is thymidine kinase.

7. The genetic construct of claim 5 wherein the selection marker is an antibiotic resistance gene.

8. The genetic construct of claim 7 wherein the antibiotic is neomycin.

9. The genetic construct of claim 1 which comprises a neomycin resistance gene, a coding sequence for chloramphenicol acetyltransferase, and a coding sequence for thymidine kinase, wherein expression of the human preproinsulin, the neomycin resistance gene, and the chloramphenicol acetyltransferase are under control of a pig preproinsulin promoter.

10. A transgenic mammal comprising a genetic construct comprising a coding sequence for human preproinsulin under control of a pig preproinsulin promoter.

11. The transgenic mammal of claim 10 which is a pig.

12. The transgenic mammal of claim 10 which is homozygous for human preproinsulin.

13. The transgenic mammal of claim 10 which is heterozygous for human preproinsulin and mammalian preproinsulin.

14. A tissue preparation obtained from a transgenic mammal comprising a genetic construct comprising a coding sequence for human preproinsulin under control of a pig preproinsulin promoter.

15. The tissue preparation of claim 14 which comprises pancreatic tissue.

16. A preparation of cells obtained from a transgenic mammal comprising a genetic construct comprising a coding sequence for human preproinsulin under control of a pig preproinsulin promoter.

17. The preparation of claim 16 which comprises fibroblasts.

18. The preparation of claim 16 which comprises islets cells.

19. The preparation of claim 16 which comprises beta cells.

20. The preparation of claim 16 which comprises stem cells.

21. The preparation of claim 20 wherein the stem cells are embryonic.

22. The preparation of claim 20 wherein the stem cells are adult.

23. A method of providing insulin or C-peptide to a patient, comprising transferring to a patient in need thereof a preparation selected from the group consisting of:
(a) pancreatic tissue obtained from a transgenic mammal comprising a genetic construct comprising a coding sequence for human preproinsulin under control of a pig preproinsulin promoter;
(b) islet cells obtained from the transgenic mammal;
(c) beta cells obtained from the transgenic mammal;
(d) stem cells obtained from the transgenic mammal;
(e) embryonic stem cells obtained from the transgenic mammal; and
(f) adult stem cells obtained from the transgenic mammal, whereby the patient's dependence on an exogenous source of insulin is decreased.

24. The method of claim 23 wherein the patient is diabetic.

25. The method of claim 23 wherein the patient has chronic pancreatitis.

26. The method of claim 23 wherein the patient has pancreatic cancer.

27. The method of claim 23 wherein the preparation is encapsulated.
